(19) United States
(12) Patent Application Publication
(10) Pub. No.: US 2014/0227370 A1
(43) Pub. Date: Aug. 14, 2014

(54) POLYSACCHARIDE FIBRES FOR WOUND DRESSINGS

(71) Applicants: Mohsen Miraftab, Wilmslow (GB); Rashid Masood, Faisalabad (PK)

(72) Inventors: Mohsen Miraftab, Wilmslow (GB); Rashid Masood, Faisalabad (PK)

(73) Assignee: University of Bolton, Lancashire (GB)

(21) Appl. No.: 14/350,174

(22) PCT Filed: Dec. 5, 2012
(86) PCT No.: PCT/GB2012/053011

§ 371 Date: Apr. 7, 2014

(30) Foreign Application Priority Data
Oct. 5, 2011 (GB) 1117140.2

(51) Int. Cl. A61K 47/36; D01D 10/06; D01D 5/06; A61K 33/38

(52) U.S. Cl. CPC A61K 47/36; A61K 33/38; D01D 10/06; D01D 5/06
USPC 424/619; 536/3; 424/618; 264/186

(57) ABSTRACT

A polysaccharide fibre useful in biomedical applications such as wound management is made as a bicomponent fibre with alginate and psyllium polymers. An antimicrobial silver salt may be incorporated. The fibre may be made by extruding an aqueous mixture of alkaline-solubilised psyllium and sodium alginate into a calcium chloride bath.

The present invention relates to polysaccharide fibres containing polysaccharide polymer derived from Psyllium husk and silver salt. The fibre of the present invention is particularly useful for biomedical applications such as wound management.

Increasing demand for special fibres produced from natural polymers is a function of their unique properties and growing areas of application. Polysaccharides are biologically produced materials that have a unique combination of functional properties and environmentally friendly features. Polysaccharides are polymers with long, chainlike structures. They provide good mechanical properties for applications as fibres, films, adhesives, thickeners, hydrogels, drug delivery agents, emulsifiers, etc. They are natural materials, produced from other biological compounds, and generally are non-toxic and biodegradable. These features make these polysaccharide materials a natural fit for sustainable development. These polysaccharides have also been considered promising materials for healthcare due to their biocompatibility, non-toxicity as well as their ease of use and ability to be easily fabricated into many different forms of products.

Psyllium is a natural polysaccharide obtained from the plantago ovata plant and has been used in many herbal remedies. It is a white fibrous material, partially soluble, hydrophilic in nature and has bulk laxative properties due to its 20 fold swelling capability when brought in contact with water, and forms a gelatinous mass. Its gelatinous mass is soluble in a dilute alkaline solution and re-gels upon acidification.

Psyllium has been widely used in the food and healthcare industry particularly for lowering cholesterol and to promote regular bowel function. FDA has also acknowledged the properties of psyllium as another significant dietary fibre that could affect blood lipids and lower the risk of Coronary Heart Disease.

Alginate is another natural polysaccharide that is produced commercially from seaweed. Over the last two decades, alginate fibres have become well established in the wound management industry where their ion exchange and gel forming abilities are particularly useful in the treatment for exuding wounds. It is biocompatible, biodegradable material and non-toxic to human body. Courtesy of its ionic substituents, alginate possesses very unique and useful properties. In particular, sodium alginate is water soluble while its calcium salt is water insoluble, although highly swelled. Conversion between the two salt forms is reversible by any ion exchange process. Aqueous sodium alginate solutions can be mixed with other materials. This material, either in wet form or after drying is very useful and can be utilized in different biologically controlled processes for specific purpose. Alginate fibres are commonly made by extruding sodium alginate solution into a calcium chloride bath, producing calcium alginate fibres.

The calcium alginate fibre is also porous material and can also be used as drug delivery agent. Calcium alginate can be used to immobilize and protect an active ingredient for storage and release under subsequent conditions. Similarly, calcium alginate can hold a component in an insoluble form while in use. It can be dissolved away when no longer required in the insoluble form. Alginate is an excellent delivery agent with valuable material properties due to its polymeric character, such as mechanical strength and adhesion.

Both alginate and psyllium are hydrophilic in nature and have intrinsic water absorbing properties, especially psyllium which has promising water absorbing properties and absorbs more than 50% of its own weight. There have been a number of reports of psyllium having wound healing properties in addition to water absorption and cholesterol lowering that would make psyllium a potentially viable commercial product. One of the main advantages that psyllium has compared to commercially available water absorbing materials is its low cost and the abundant availability of the raw material. The present invention is based on the realisation that a conjugate fibre of alginate and psyllium that has good textile processability, and is of uniform composition along the length of the produced fibres can be made at a lower cost than other commercially available products.

The property of gel fraction solubility of psyllium in dilute alkaline solution and re-gelling with acid has shown its suitability for use with other fibre forming polysaccharides especially alginate.

According to one aspect of the invention therefore, there is provided a polysaccharide fibre having, as components of the same fibre, alginate and psyllium polymers.

Preferably, the alginate and psyllium polymers are bonded to each other. Thus, the fibre may be produced by extrusion or spinning of a mixture of the alginate and psyllium polymers.

The fibre may be a bicomponent fibre i.e. containing the psyllium and alginate polymers as the only structural components. Alternatively other structural components may be incorporated.

The fibres of the invention can be used in biomedical applications, particularly wound management and may have improved tensile properties and superior liquid absorbency compared to pure alginate fibres.

Preferably the fibre also incorporates an antimicrobial substance or substances, particularly silver substances. Thus, and in accordance with a second aspect of the invention, which may also incorporate any or all features of the first aspect, there is provided an antimicrobial fibre containing alginate, psyllium and a silver substance. In this way, a truly antimicrobial conjugate fibre with excellent absorption properties can be made.

The silver substances are preferably incorporated as a water soluble compound i.e. silver nitrate and a partially soluble organic silver compound i.e. silver carbonate.

Most preferably the alginate is incorporated in the form of a water soluble alginate, particularly sodium alginate, and the fibre is formed by extrusion into a coagulating bath which converts the alginate to an insoluble salt, particularly a calcium chloride bath.

In accordance with a further aspect of the invention there is provided a method of making a polysaccharide fibre comprising spinning or extruding an aqueous mixture of soluble psyllium and alginate polymers into a bath containing a substance which coagulates the aqueous mixture to form a fibre.

The procedure of the invention may comprise the steps of extruding a solution of sodium alginate, psyllium and silver compounds (silver nitrate and silver carbonate) directly into a coagulation bath of 1-2% calcium chloride. The conjugate fibre thus formed can then be washed by being passed through a water bath followed by a number of baths containing acetone-water mixtures to bring about water-solvent exchange within the fibres. The purpose of this step is to facilitate drying of the fibres using hot air, after which the fibres are wound up and may be further processed into non-woven felts or ropes or other structures commonly used for wound management.

The first stage of the fibre production process is to have a controlled degree of purification of psyllium gel. The effect of heat is to cause both depolymerisation and solubilisation to a controlled degree.

It is not feasible to extrude alginate/psyllium fibres in raw form because of Psyllium/husk contamination and un-dissolved contents of raw Psyllium. Therefore, First psyllium gel extraction have been made using cold water, hydrolysis with a base such as sodium hydroxide and heat may be used, in order to fully dissolve the psyllium for extrusion with alginate.

Although a range of available sodium alginates could be used, the preferred sodium alginate used to produce the conjugate fibres of the invention is Protanal LF 10/60 (supplied by Pronova, Norway) with a high guluronic ratio (ManA 25-35%, GulA 65-75%) or solutions of varying concentration may be used, typically 1 to 6% (w/v), more preferably 2-5% (w/v), most preferably 3-4% (w/v).

The sodium alginate and psyllium polymer solutions are typically ejected under pressure of around 2.2 bar through a spinneret having a number of apertures of defined diameter e.g. 40 to 200 holes with an average diameter of 50 μm into a coagulation bath of 1-2% Calcium Chloride. The alginate and psyllium dope is delivered into the 1-2% Calcium Chloride bath at the rate of ˜5 cm³/min and drawn up to 100% by pick up rollers before being washed in normal water and dried by passing through baths of acetone-water mixtures with increasing concentrations of acetone in each bath i.e. the first bath contains 50% acetone, the second 70% acetone and the final bath 100% acetone. This enables the fibres to be dried using hot air (60-80° C.) as they are wound onto a spool.

The properties of fibres produced by this method are found to have improved absorption characteristics when compared to calcium alginate and to be stronger in terms of force to break in comparison with calcium alginate. The tenacity of alginate/psyllium fibres produced according to the method of this invention lies in the range between 2.8 to 11.5 cN/tex, whereas typical values for calcium alginate fibres are between 2-10 cN/tex.

The absorption of alginate/psyllium composite fibres produced according to the method of this invention lies in the range from 16-25 (g/g) for water and 25-35 (g/g) for saline. The fibres diameter also enlarges dramatically upon exposure to either water or saline solutions. Corresponding values for calcium alginate fibres are 6-10 (g/g) water and 9-12 (g/g) saline.

The produced fibres have distinctive morphological characteristics i.e. soft surface with near perfect uniformity resembling surface characteristics of silk fibres. These features are distinctly different to those of pure alginate.

Furthermore, these properties can also be manipulated by including other additives to enhance performance and functional requirements. Examples of additives that may be incorporated into the fibres of the invention either during the production of the fibres or by subsequent post treatment are antimicrobial agents such as silver ions, chlorhexidine or any antibiotic drug, agents known to influence wound healing such as zinc ions, aloe vera or salts of hyaluronic acid and fragrances such as lavender or oil of rosemary.

The invention will now be described by means of the following non-limiting examples.

### EXAMPLE 1

3-6 g of psyllium was soaked in 1 litre of deionised water for 2 hours. The soaked psyllium was heated up to boiling point for 40-60 minutes. The solution was stirred and filtered in order to separate the un-dissolved particles. 4-4.5% sodium alginate was added and stirring continued for a further 4 hours. The solution was then extruded into 1-2% Calcium Chloride. Extruded fibres were drawn, washed in cold water and passed through acetone water mixtures of increasing acetone concentrations i.e. 50-100% before being dried by hot air. The properties of the fibres when tested were tenacity of 3.3-6.0 cN/tex, elongation at break 5.3-12.4%, water absorption 18-25 g/g and saline absorption 20-30 g/g.

### EXAMPLE 2

10-15 g of psyllium was soaked in 1 litre of deionised water with 0.1%-0.5% NaOH for 2 hours. The soaked psyllium was heated up at low temperature 50-60° C. for 20-30 minutes. The solution was stirred and filtered in order to separate the un-dissolved particles. 4-4.5% sodium alginate was added and stirring continued for a further 4 hours. The solution was then extruded into 1-2% Calcium Chloride. Extruded fibres are drawn, washed in cold water and passed through acetone water mixtures of increasing acetone concentrations i.e. 50-100% before being dried by hot air. Recorded fibre properties were more or less similar to those in example one.

### EXAMPLE 3

5-15 g of psyllium was soaked in 1 litre of deionised water for 1-2 hours. The soaked psyllium was stirred for 1 hour and filtered in order to separate the un-dissolved particles. 4-4.5% sodium alginate was added and stirring continued for a further 4 hours. The solution was then extruded into 1-2% Calcium Chloride. Extruded fibres are drawn, washed in cold water and passed through acetone water mixtures of increasing acetone concentrations i.e. 50-100% before being dried by hot air. The properties of the fibres when tested were tenacity of 5.5-11.0 cN/tex, elongation at break 7.1-11.4%, water absorption 18-25 g/g and saline absorption 20-33 g/g.

### EXAMPLE 4

3-6 g of psyllium was soaked in 1 litre of deionised water for 2 hours. 0.12-0.60 g of silver nitrate was dissolved in 200 ml and boiled for 20-30 minutes separately. The soaked psyllium was added to boiling silver nitrate solution and left on the heat for another 30 minute while stirring. The solution was stirred and filtered in order to separate the un-dissolved particles. 3-4% sodium alginate was added and stirring continued for a further 4 hours. The solution was then extruded into 1-2% Calcium Chloride. Extruded fibres were drawn, washed in cold water and passed through acetone water mixtures of increasing acetone concentrations i.e. 50-100% before being dried by hot air. Recorded fibre properties are similar to those in Example 1.

### EXAMPLE 5

5-15 g of psyllium was soaked in 1 litre of deionised water for 1-2 hours. The soaked psyllium was stirred for 1 hour and filtered in order to separate the un-dissolved particles. 0.12-1.00 g of silver carbonate was added and stirring continued for another hour. 4.0-4.5% sodium alginate was added and stirring continued for a further 4 hours. The solution was then extruded into 1-2% Calcium Chloride. Extruded fibres are drawn, washed in cold water and passed through acetone water mixtures of increasing acetone concentrations i.e. 50-100% before being dried by hot air. The properties of the fibres when tested were tenacity of 4.19-12.0 cN/tex, elongation at break 7.1-10.4%, water absorption 18-25 g/g and saline absorption 20-28 g/g.

The invention is not intended to be restricted to the details of the above Examples.

1. A polysaccharide fibre having, as components of the same fibre, alginate and psyllium polymers.

2. A fibre according to claim 1 wherein the alginate and psyllium polymers are bonded to each other.

3. A fibre according to claim 1 which is produced by extrusion or spinning of a mixture of the alginate and psyllium polymers.

4. A fibre according to claim 1 which is a bicomponent fibre containing the psyllium and alginate polymers as the only structural components.

5. A fibre according to claim 1 further including an antimicrobial substance.

6. A fibre according to claim 5 wherein the antimicrobial substance is a silver substance.

7. An antimicrobial fibre containing alginate, psyllium and a silver substance.

8. (canceled)

9. A fibre according to claim 6 wherein the silver substance is silver nitrate or silver carbonate.

10. A method of making a polysaccharide fibre comprising spinning or extruding an aqueous mixture of soluble psyllium and alginate polymers into a bath containing a substance which coagulates the aqueous mixture to form a fibre.

11. A method according to claim 10 wherein the coagulating substance comprises a calcium salt which forms insoluble calcium alginate with the soluble alginate polymer of the aqueous mixture.

12. A method according to claim 11 wherein the bath contains 1-2% by weight calcium chloride.

13. A method according to claim 10 wherein the soluble psyllium polymer is solubilised in alkaline solution.

14. A method according to claim 10 wherein the soluble alginate polymer comprises sodium alginate.

15. A method according to claim 10, further including washing the formed fibre with aqueous acetone.

16. A method according to claim 10 wherein the aqueous mixture contains psyllium and alginate polymers as the sole structural polymers.

17. A method according to claim 10 further including an antimicrobial substance in the aqueous mixture.

18. A method according to claim 17 wherein the antimicrobial substance is a silver substance.

19. A method according to claim 18 wherein the silver substance is silver nitrate or silver carbonate.

20. A fibre formed by the method of claim 10.

21. (canceled)

22. (canceled)
